(19) United States
(12) Plant Patent Application Publication
(10) Pub. No.: US 2002/0129416 P1
(43) Pub. Date: Sep. 12, 2002

(54) CHRYSANTHEMUM PLANT NAMED 'COOL TIME'

(76) Inventor: Mark Roland Boeder, West Wittering (GB)

Correspondence Address: Mark K. Dickson, HOWREY SIMON ARNOLD & WHITE, LLP, 750 Bering Drive, Houston, TX, 77057-2198, US

(21) Appl. No.: 09/802,051

(22) Filed: Mar. 7, 2001

(51) Int. Cl. A01H 5/00

(52) U.S. Cl. PLT/294

(57) ABSTRACT

A new variety of Chrysanthemum plant named ‘Cool Time,’ with bright white, single type flowers.

### BACKGROUND OF THE INVENTION

The present invention comprises a new and distinct cultivar of Chrysanthemum botanically known as Dendranthema grandiflora, and referred to by the cultivar name ‘Cool Time’.

‘Cool Time’, identified as ‘COT1’ originated from a cross made by Mark Boeder in a controlled breeding program at Chichester, West Sussex, United Kingdom. The female parent of the new variety is an undistributed, unpatented seedling identified as 90547 and the male parent of the new variety is an undistributed, unpatented seedling identified as 90529. The new variety ‘Cool Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Cool Time’ is a pot type of Chrysanthemum plant variety having bright white single-type flowers.

### Comparison with Parent

Plants of the new Chrysanthemum variety ‘Cool Time’ are similar to the male parent ‘90529’ in flower colour and response time. In side-by-side comparisons at Chichester, West Sussex, United Kingdom, under commercial practice, plants of the new Chrysanthemum variety ‘Cool Time’ differed from plants of the female parent ‘90547’ in the following characteristics.

1. The new variety ‘Cool Time’ produces single type flowers whereas the parent cultivar ‘90547’ produces anemone type flowers.

2. Plants of the new variety ‘Cool Time’ have larger flowers than plants of the parent cultivar ‘90547’.

### Comparison with Other Varieties

Plants of the new Chrysanthemum variety ‘Cool Time’ are similar to the unpatented cultivar ‘Tasman’ in flower colour and flower type. However, in side-by-side comparisons at Chichester, West Sussex, United Kingdom, under commercial practice, plants of the new Chrysanthemum variety ‘Cool Time’ differed from plants of the cultivar ‘Tasman’ in the following characteristics.

1. The new variety ‘Cool Time’ produces pure white flowers whereas the cultivar ‘Tasman’ produces cream-white flowers.

2. Plants of the new variety ‘Cool Time’ have larger flowers than plants of the cultivar ‘Tasman’.

### BRIEF DESCRIPTION OF ILLUSTRATIONS

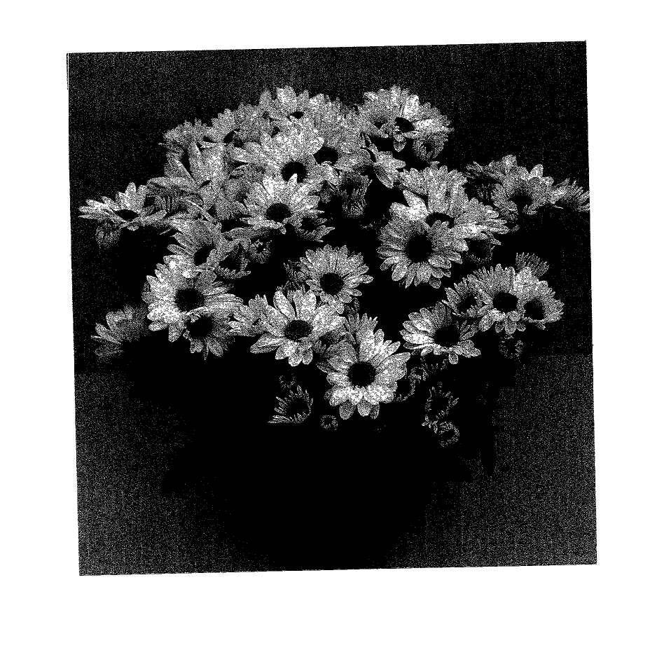

Typical specimens of the plant and flowers for the new Chrysanthemum variety ‘Cool Time’ are shown in the accompanying photograph. The colors shown are as true as possible within the usual limits of this kind of illustration. FIG. 1 is a whole plant view of the new variety ‘Cool Time’ grown in a pot.

### DETAILED BOTANICAL DESCRIPTION

The following description of the new Chrysanthemum variety ‘Cool Time’ is of plants grown in a greenhouse at Chichester, West Sussex, United Kingdom in the month of December. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 18° C. to 24° C. during the day under light conditions of about 5,000 to 6,000 foot candles. The plants respond well to the use of growth retardant, such as three B9 treatments at about 2 gram/liter. To produce a commercial product the plants may be pinched once. The typical container size for commercial growth is 1 liter. It has been observed that the shelf life of the new variety is about 20 days with a response time of about 7.5 weeks.

The new variety may be produced as a spray. The following description is with respect to a plant produced as pot Chrysanthemum. In the description of this new Chrysanthemum variety, color values have been taken from The Royal Horticultural Society Colour Chart (R.H.S.C.C.).

### Plant

Plant type: pot chrysanthemum

Habit: upright

Height: 20-30 cm

Branching characteristics (describe): upright spreading

Length of Lateral Branches: 13-18 cm

Number of Breaks from Pinch: 3-5

Stem Color: near 147C

Response Time: near 7.5 weeks

Vigor: medium

Shelf Life: near 20 days

Disease (susceptibility or resistance observed): no observations

Growth retardant type and treatment: 3 applications B9 (2 gram/liter)

Propagation:

Type.—vegetative propagation via stem cuttings.

Time to rooting.—14 days with soil temperatures of 18° C.

Rooting habit.—After 7 Long Days (20 hrs light) first roots emerge from root primordia. After 14 Long Days a complete root system is developed.

### Foliage

Number of Leaves Per Lateral Branch: 7-10

Compound or single: single

Arrangement of Leaves: alternate

Shape of leaf.—typical 5 lobed, oblong.

Size of leaf.—Width (cm): near 4.0-6.0 Length (cm): near 8.0-11.0.

Leaf Apex.—mucronate.

Base.—cordate.

Attachment.—petioled.

Aspect.—slightly undulating.

Margin.—palmately lobed.

Surface characteristics.—Top: pubescent Bottom: pubescent.

Petiole:

Color.—near 137C.

Length.—near 2.0-3.0 cm.

Venation (describe): prominent midvein

Color.—upper side: near 138D under side: near 138D.

Color:

Mature leaf, upper side.—near 137A; under side: near 137C.

Young leaf, upper side.—near 137B; under side: near 138B.

### Flower

Flower type: single

Flower form: flat ray florets and tubular disk florets

Number of blossoms per branch: 4-6

Inflorescence form: cyme

Depth of fully expanded blossoms.—near 2.0-3.0 cm.

Diameter of fully expanded blossoms.—near 7.0-9.0 cm.

Sepals:

Number.—20-30.

Color.—near 147A.

Length.—0.4-1.0 cm.

Width.—0.1-0.4 cm.

Texture/Appearance.—pubescent.

Peduncle length is evenly distributed along the stem; longest length is on average 4.0 cm, shortest length is on average 1.0 cm:

Length.—1.0-4.0 cm.

Color.—near 147C.

Surface.—pubescent.

Habit.—slight undulating.

Strength.—medium.

Pedicel:

Length.—terminal: 11.0-14.0 cm; lateral 13.0-17.0 cm.

Color.—near 147B.

Surface.—pubescent.

Habit.—slight undulating.

Strength.—medium.

Ray Florets:

Form/Shape.—flat.

Texture/Appearance.—matte.

Number per flower.—25-35.

Length.—near 2.5-3.5 cm Width: near 0.5-1.0 cm.

Apex.—rounded.

Base.—tapered.

Margin.—entire.

Disc florets:

Form/Shape.—tubular.

Texture/Appearance.—matte.

Number per flower.—180-240.

Length.—near 0.4-0.6 cm; Width near 0.1 cm.

Diameter of disc.—near 1.2-2.0 cm.

Fragrance: very faint

Flower Bud (at onset of color):

Length.—0.5-0.8 cm.

Diameter.—0.9-1.3 cm.

Form/shape.—spherical.

General flower color:

1. Ray florets, upper side.—Immature: near 155A Mature: near 155D Older/Fading: near 155D.

2. Ray florets, under side.—Immature: near 155A Mature: near 155D Older/Fading: near 155D.

3. Disc florets.—Immature: near 147B Mature: near 151A Older/Fading: near 151C.

4. Bud.—near 147B.

Flower progression with age: rayflorets open cream white and change to pure white flower colour.

Other Distinguishing Characteristics: Pollen is visible on stigma in early stage

### Reproductive Organs

Ray Florets: Pistillate

Pistil number.—about 5.

Stigma color.—near 12A.

Stigma shape.—fork.

Style color.—near 149C.

Style length.—0.3-0.7 cm.

Disc Florets: Pistillate

Pistil number.—about 5.

Stigma color.—near 12A.

Stigma shape.—fork.

Style color.—near 149C.

Style length.—0.2-0.6 cm. Staminate:

Stamen number.—about 5.

Anther shape.—thread.

Anther color.—near 13B.

Pollen color.—near 13B.

Pollen quantity.—little.

What is claimed is:

1. A new and distinct variety of Chrysanthemum plant, substantially as described and illustrated herein.
